(19) United States
(12) Patent Application Publication
(10) Pub. No.: US 2012/0085363 A1
(43) Pub. Date: Apr. 12, 2012

(54) TREATING LICE WITH GASEOUS COMPOUNDS IN AN AIRTIGHT SPACE

(75) Inventors: Menachem RITTERBAND, Nes Zionna (IL); YINON SHLOMI, Rehovot (IL)

(73) Assignee: NOVOMIC LTD, Ramat Gan (IL)

(21) Appl. No.: 12/901,544

(22) Filed: Oct. 10, 2010

(51) Int. Cl. A61K 8/18; A45D 19/16

(52) U.S. Cl. 132/202; 132/212

(57) ABSTRACT

A system and a method for treating lice and nits on a head of a patient. The system includes a cap for defining an airtight space around the head and a container for storing a mixture of gases for treating lice, a gas release mechanism for delivering the mixture of gases into the airtight space as an aerosol, and a gas distribution device having plurality of gas delivery pins. The mixture of gases causes irreversible damage to essential mechanisms of the lice. According to another embodiment of the present invention the system includes a cap for defining an airtight space around the head and an active agent container. The active agent of the type of acetic acid, stored in a liquid state, and vaporized during use, then circulated toward the cap.

### REFERENCE TO CROSS-RELATED APPLICATION

This application is a continuation-in-part of U.S. Patent Application No. 20090235949 filed on May 27, 2009 which is a continuation-in-part of International Application No. PCT/IL2008/000031 filed Jan. 6, 2008.

This application claims priority benefits from U.S. Patent Application No. 20090235949 filed on May 27, 2009 which claims priority benefits from PCT/IL2008/000031 filed Jan. 6, 2008, which claims priority benefits from U.S. Provisional Application No. 60/878,351, filed on Jan. 4, 2007, herein incorporated by reference in its entirety.

### FIELD OF THE INVENTION

The present invention relates to the field of medical treatment systems, more particularly, to a system and method for treating an infestation of head lice.

### BACKGROUND OF THE INVENTION

Infestation of the human body by lice is an increasingly prevalent social and health problem in many countries, including the USA. Lice infest hundreds of millions of people each year. Lice are very small insects, about 2-3 mm in length. They deposit eggs either on a hair or fabric fiber and attach them firmly with a cement-like excretion.

The life cycle of lice comprises an egg stage, three nymphal stages, and an adult stage, and takes about one month. A female louse lays approximately 100 eggs, which are glued to hairs of the host. The eggs generally hatch in about six to ten days, depending on temperature. The nymphs, (the larval stage in insect development), and adults suck blood, causing disturbance (itching, also known as pruritus) and secondary infection. The empty shells remaining after the nymphs emerge from the eggs look like white grains of sand.

Lice, being insects, use tracheae for respiration. Tracheae are a system of internal tubes (invaginations of the cuticle) penetrating the insect's body, through which air diffuses or is being pumped directly to the body tissues. Within body cells, chemical respiration takes place in the mitochondria, where ATP is produced under oxygen consumption.

The common treatment methods against lice involve liquids or lotions. An apparatus and process for killing human and animal vermin, which discloses treatment of lice within a gas-tight garment by using a volatile liquid, specifically methyl-formate, that is spread on a pad inside the garment and evaporates inside the garment, is described in U.S. Pat. No. 2,033,357 of Riker et al. patented 1936.

A method and device for eradicating lice and nits form an infested area, particularly the scalp of a human being, comprises a plastic or rubber cap for covering the head of a human being, and means for filling the cap with an inner gas, such as carbon dioxide from a pressurized-gas cartridge, in order to suffocate the lice and the nits enclosed by the cap, is described in EP Patent Application No. 8663016.9, publication No. 022100a4 A2, of Scolnik et al. filed Oct. 30, 1986.

A formulation for controlling human lice, which discloses pediculicidal formulations comprising a spinosyn, or a physiologically acceptable derivative or salt thereof, and a physiologically acceptable carrier, and methods of controlling lice infestations in a human with these formulations are provided in International Application No. PCT/US1999/013925, Publication No. WO/2000/001347, of Snyder, filed Jun. 21, 1999.

A pediculicide composition, in particular, a composition comprising a sesquiterpene alcohol and a suitable carrier, is described in International Application No. PCT/AU2006/001720, Publication No. WO/2007/056813, of Found, filed Nov. 17, 2006.

A method and composition for controlling lice on an organism is described in International Application No. PCT/NL2000/000196, Publication No. WO/2000/057704, of Kussendrager et al. filed Mar. 23, 2000.

All of these applications are incorporated by reference for all purposes as if fully set forth herein.

Although the various breeds of human lice are related, each of them has specific characteristics with regard to habitat and feeding. For example, head lice are small hard-shelled ectoparasites which cling to hair shafts while feeding, mating and laying eggs. The louse must remain on the head or it will die within a short period of time. Head lice proliferate at an incredible rate. A louse is ready to mate and reproduce within 10 hours after hatching. Under ideal conditions, a female louse may produce up to 300 eggs in its lifetime. Ideal conditions include an adequate food supply, environmental temperatures from about 28° C. to about 32° C., and relative humidity of about 70% to about 90%.

The louse's hard chitinous exoskeleton serves as protection from external elements. Lice eggs (or ova) are similarly protected by a chitinous sheath surrounding the eggs and attached to the hair shaft. Although lice may be affected by the use of an insecticide, the eggs often remain resistant to attack. Thus, present art optimum treatment of a lice infestation includes both a pediculicide, which kills the adult lice, and an ovicide, which interrupts the development of the eggs.

Treatment for eliminating head lice traditionally included home remedies such as smearing mayonnaise, olive oil, hair pomade, or some other heavily viscous material about an infested scalp coupled with rigorous combing of the hair and meticulous removal of adult lice, nymphs, and nits. Though these home remedies do not kill head lice, the prevailing thought is that the viscosity of the material makes it hard for head lice to roam about the scalp, making for easy removal. Such home remedies are usually ineffective at controlling head lice due to the ability of the lice to revive rapidly once these materials are removed.

More effective treatments for eliminating head lice involve massaging the infested scalp with over-the-counter (OTC) topical creams containing active insecticides. Because of their potential toxicity to the human host, the use of these topical formulas is regulated by the FDA. Over-the-counter insecticides typically have pyrethrins or permethrin as active ingredients

Biologically active agents have been used for some time in attempts to control lice. For example, lindane (gammabenzene hexachloride), organophosphates (malathion), natural pyrethrins, and synthetic compounds known as pyrethroids (such as permethrin) have been used as pediculicides in lice treatment formulations. These agents however, have drawbacks. Lindane has a poor safety profile, and lice have developed resistance to it. Natural pyrethrin requires frequent follow-up treatments because it provides only short term residual action. Synthetic pyrethroids, although more effective against lice than natural pediculicides, are often more toxic to the subject being treated.

Strains of head lice have been identified worldwide which are resistant to all currently available topical treatments. Possible neural damage to the human host prevents raising the insecticide levels above the current threshold in an attempt to combat these newer treatment resistant head lice.

None of the prior art provides a sufficiently effective solution, and there is therefore a need for a system and a method for treating an infestation of head lice.

### SUMMARY OF THE INVENTION

The present invention teaches an effective solution for an infestation of head lice, including a system and method of use, which eliminates both the lice and their eggs by means of effective insertion of gas near the roots of the patient's hair, with the gas including toxic material or materials, and enabling a combined attack also including suffocating gases.

The system for treating lice and nits on a head of a patient includes a cap for defining an airtight space around the head and a container for storing a mixture of gases and compressed gases for treating lice. In addition, a gas release mechanism for delivering the mixture of gases into the airtight space is also provided. Specifically, the mixture of gases is selected so that it causes irreversible damage to at least one essential mechanism of said lice.

According to the present invention an effective concentrations of acidic vapors in general and Carboxylic acids in particular are applied. The invented treatment is based on the realization that live lice and their eggs are damaged by inhalation or absorption of reactive acidic gas or vapor. Hence the present invention includes compositions comprising of carboxylic acids and methods of their application to human subjects.

While prior art methods involve application of active ingredients in liquid or cream forms, the present invention specifies the application of carboxylic acids active ingredients in gas or vapor form. Moreover, for containment of said gas or vapor near the scalp, said gas or vapor application is introduced under a head cap covering the hair and relatively well sealed under the hair line by snug fit of an elastic perimeter of said head cap.

Folk art of applying various substances to the head for controlling head lice sometimes suggest applying a covering cap on the head after a shampoo, gel, or liquid is applied onto the scalp and hair. In contrast, the method, according to the present invention, demands prior covering of the head with a covering cap and then after introducing the gas or vapor composition comprising of carboxylic acid into the space under said cap. That introduction is preferably done via dedicated passages incorporated into said cap.

The process of evaporation in a closed container will proceed until there are as many molecules returning to the liquid as there are escaping. At this point the vapor is said to be saturated, and the pressure of that vapor (usually expressed in mmHg) is called the saturated vapor pressure. At room temperature, the saturated vapor pressure is the maximum partial pressure concentration at which the gas can be obtained in gaseous form.

The overall concentration of each of said carboxylic acids components in the applied gas or vapor compositions is selected to be on the one hand effective in reducing the survival of lice and/or eggs and on the other hand to be of tolerable irritability to the skin of human subjects. Effective concentrations satisfying the above constrains, in part from room temperature saturated vapor saturation pressure, can be selected for example from: between 1% to 5%, between 5% to 10%, between 10% to 20%, between 20% to 30%, between 30% to 40%, between 40% to 50%, between 50% to 60%, between 60% to 70%, between 70% to 80%, and between 80% to 90%.

In some embodiments, a mixture of more than one type of carboxylic acid is contained in the composition according to the present invention. An advantage of such a mixture is that, since each type of carboxylic acid is active in deactivating lice, and since the maximum gas concentration of each carboxylic acid is limited by its associated saturation partial pressure at room temperature, the use of a mixture of several carboxylic acids can produce an overall increase in the total vapor concentration of effective carboxylic acids active ingredients.

One type of gas or gas mixture may target the respiratory system by attacking the soft tissues or by elimination of oxygen from binding to the oxygen carrying protein of the lice.

One type of gas or gas mixture may target the respiratory system by attacking the soft tissues (for example vapors of acids such as acetic acid, hydrochloric acid and nitric acid), or by elimination of oxygen from binding to the oxygen carrying protein of the lice (for example carbon monoxide).

Other gasses targeting other targets such as the reproductive system (e.g. some of the organophospates), or nervous system(e.g. insecticides belonging to organochlorines, carbamates, and organophosphates) may be combined at the same time, before, or after treatment with the first gas mixture. The combination of gasses for targeting two or more different systems may improve the effectiveness of treatment.

In some embodiments treatment is done first with one type of gas and afterwards with another type of gas, for example, the lice are initially attacked with toxic gas which diminishes their resilience, and afterwards they are attacked with suffocating gas. This way, the use of toxic gas can be reduced.

According to the present invention, the systems of the lice that are attacked include: the respiratory system, by attacking mucous membrane tissue; the respiratory system, by attacking joints; the respiratory system, by disrupting the body's absorption of oxygen; the nervous system; and the reproductive system.

The dilution of the carboxylic acid active ingredient in the applied compositions may be in liquids such as water or in other gases such as air or water vapor, but these examples should not be taken as limiting.

It is a core principle of the present invention that, while the storage for of the compositions may be in liquid or compressed gas form, the release and application of said composition onto the proximity of the human head subject is in gas or vapor form.

Processes of creating a carboxylic acid gas or vapor from a storage compartment can be achieved in many forms, also including: reaction of high concentration acid with water, and compressed gas containers.

A particular benefit of the formulations is their effectiveness on both lice and eggs, and against louse species that have become resistant to currently used products.

Another benefit of the present invention is that there is no need for intensive washing of the hair after application of the treatment.

Carboxylic acids are known as a general family of chemicals representative members of which are selected from the group comprising of: formic acid (insect stings), acetic acid, propionic acid, butyric acid, valeric acid (valerian), caproic acid, enanthic acid, caprylic acid, pelargonic acid, capric acid, lauric acid, palmitic acid, and stearic acid.

Experiments have shown that the carboxylic acids most effective for use according to the present invention are formic acid and acetic acid.

Formic acid, HCOOH or CH₂O₂ is the simplest carboxylic acid.

Acetic acid, CH₃COOH, or C₂H₄O₂, also known as is an organic acid, is a simple carboxylic acid too, which is a water-soluble component of vinegar, and has been used, in liquid, as a folk remedy which dissolves the cement of lice eggs.

In some embodiments, a vapor mixture of carboxylic acid in air is first produced in a closed container of volume V_{con}. Said closed containers can be selected from the group of: bottles, bags, balloons, or other containers known in the art. According to the present invention, the gas from said container is to be delivered into the space between the head cap and the patient scalp. It is assumed that there is some desired predetermined vapor concentration Cᵥ of a selected carboxylic acid, in order to create an effective formulation according to the present invention.

The production for such vapors from solid or liquid forms of carboxylic acids can be done in the following production process: (a) A thin film or powder of a carboxylic acid is first enclosed between two strips or plates to create a “strip source”. The strip source is placed inside a container. (b) The quantity of carboxylic acid contained in the strip source is selected such that if it is evaporated within said selected container with volume V_{con} then it can create a vapor of said carboxylic acid with at least with the desired concentration Cᵥ. (c) The strip source is placed inside the selected container. (d) To create the carboxylic acid vapor at any prescribed or desired time, the two sides of the strip source are separated so as to reveal the included film or power of carboxylic acid to contact with the surrounding air within said container. (e) The container is then kept sealed at least for the amount of time that is required for sufficient evaporation of said carboxylic acid film or powder in the amount to create the desired concentration Cᵥ within said container.

According to an embodiment of the present invention active agent, which is in liquid state, is stored within an active agent container, absorbed by a sponge. During the treatment process, gas at a pressure higher than one atmosphere is inserted into the active agent container. This gas can be air or any other suitable gas or mixture of gases. This gas causes the evaporation of the active agent and its flow into the dispersal system contained within the cap worn by the patient.

According to an embodiment of the present invention there is provided a system for treating lice and nits on a head of a patient, the system including: an active agent container for storing a predetermined volume of active agent, wherein the active agent container includes: a container main wall having a container main wall first end and a container main wall second end; and two container end walls, disposed on the container main wall first end and the container main wall second end, wherein the system for treating lice and nits has a storage state and active state.

According to further feature in the described embodiments the system for treating lice and nits is in the storage state, wherein the active agent container store an active agent, wherein the active agent is in a liquid state.

According to still further features in the described embodiments the active agent container store a sponge, wherein the active agent is absorbed by the sponge.

According to still further feature in the described embodiments the active agent container store a plurality of sponge pieces, wherein the active agent is absorbed by the sponge pieces.

According to still further feature in the described embodiments the active agent is an Acetic acid.

According to still further feature in the described embodiments the active agent container further includes: at least on filter disposed inside the active agent container; and two caps mounted on the two container end walls.

According to still further features in the described embodiments the active agent has a volume of at least two milliliters.

According to still further feature in the described embodiments the active agent container further includes: at least one sealing disc disposed inside the active agent container.

According to another feature in the described embodiments the system for treating lice and nits is in the active state, wherein the active agent container store an active agent, wherein the active agent is partially in a liquid state and partially in a gas state.

According to further features in the described embodiments the system for treating lice and nits further including: a cap, wherein the cap is operatively connected to the active agent container.

According to still further features in the described embodiments the system for treating lice and nits further included: an inlet tube wherein the inlet tube is connected to the active agent container; and (c) a cap, wherein the cap is operatively connected to the inlet tube.

According to still further feature in the described embodiments the system for treating lice and nits further includes: (d) a vaporizing and circulating gas source, wherein the vaporizing and circulating gas source is operatively connected to the active agent container.

According to still further feature in the described embodiments the cap is made from a material that is practically impenetrable to the active agent, wherein the cap has an interior side.

According to still further feature in the described embodiments the cap includes: a distributor; at least two injection tubes, wherein each one of the injection tubes is operatively connected to the injection tubes, wherein each one of the injection tubes is disposed on the interior side of the cap, and wherein each one of the injection tubes has plurality of flow openings.

According to still further features in the described embodiments there is a flow of active agent from the active agent container through the distributor, through the injection tubes and through the flow opening.

According to still further feature in the described embodiments there is a continuous flow of vaporizing and circulating gas from the vaporizing and circulating gas source through the active agent container, at a rate of at least one litter per minute for at least three minutes.

According to an embodiment of the present invention there is provided a method for treating lice and nits on a head of a patient the method including the stages of: mounting a cap over the head of the patient, the cap includes an inlet tube, a distributor and injection tubes; connecting an active agent container to the inlet tube, wherein the active agent container includes an active agent in liquid state absorbed in a sponge; delivering vaporizing and circulating gas through the active agent container, and evaporating the active agent; waiting at least a predetermined minimal duration; and removing the cap from the head after the predetermined minimal duration.

According to further feature in the described embodiments the active agent is an acetic acid.

According to still further feature in the described embodiments there is a flow of the active agent from the active agent container, through the distributor, and through the injection tubes.

Additional objects and advantages of the invention will be set forth in part in the description which follows and, in part, will be obvious from the description, or may be learned by practice of the invention.

### BRIEF DESCRIPTION OF THE DRAWINGS

The invention is herein described, by way of example only, with reference to the accompanying drawings, wherein:

FIG. 1 illustrates a first embodiment of a system for treating lice and nits, according to the present invention.

FIG. 2 illustrates a second embodiment of a system for treating lice and nits, according to the present invention.

FIG. 3 illustrates the first embodiment of a system for treating lice and nits, on a head of a patient, having two sets of rigid containers inlet tubes and unidirectional valves, according to the present invention.

FIG. 4 illustrates the first embodiment of a system for treating lice and nits, on a head of a patient, having two sets of rigid containers, unidirectional valves, and inlet tubes, according to the present invention, with both inlet tubes joining into a single inlet tube.

FIG. 5 illustrates a third embodiment of a system, for treating lice and nits, on a head of a patient, having a gas distribution device, according to the present invention.

FIG. 6a illustrates a bottom view of a gas distribution device, according to the present invention, upon which the section plane a-a is marked.

FIG. 6b is a cross sectional view a-a schematic illustration of an exemplary, illustrative embodiment of a hollow gas distribution device, according to the present invention.

FIG. 7a illustrates the first embodiment of a system for treating lice and nits, on a head of a patient, according to the present invention, in which the active material is separately stored within an active material container.

FIG. 7b shows two active material containers, according to the present invention.

FIG. 8 is a block diagram schematically illustrates a method of treating lice and nits on a head of a patient, according to some embodiments of the invention.

FIG. 9 illustrates a fourth embodiment of the system for treating lice and nits, according to the present invention.

FIG. 10a illustrates a cap of the fourth embodiment of the system for treating lice and nits, according to the present invention, upon which a section plane b-b is marked.

FIG. 10b is a cross sectional view b-b illustration of an injection tube, of the cap of the fourth embodiment of the system for treating lice and nits, according to the present invention.

FIG. 11a is a side view schematic illustration of an active agent container of the fourth embodiment of the system for treating lice and nits, according to the present invention.

FIG. 11b is an isometric view schematic illustration of an active agent container of the fourth embodiment of the system for treating lice and nits, according to the present invention.

FIG. 11c is a top view schematic illustration of an active agent container of the fourth embodiment of the system for treating lice and nits, according to the present invention, upon which a section plane c-c is marked.

FIG. 11d is a cross sectional view c-c illustration of an active agent container of the fourth embodiment of the system for treating lice and nits, according to the present invention

FIG. 11e is an isometric view schematic illustration of a filter compartment and a filter, of the active agent container, of the fourth embodiment of the system for treating lice and nits, according to the present invention.

FIG. 12 is an isometric view schematic illustrations of a vaporizing and circulating gas source, an active agent container, and an inlet tube, of the fourth embodiment of the system for treating lice and nits, according the present invention.

FIG. 13 is a block diagram schematically illustrating of a second method of treating lice and nits on a head of a patient, according to some embodiments of the present invention.

### DETAILED DESCRIPTION OF EMBODIMENTS

The present invention is of a system and a method for treating lice and nits on a head of a patient.

The principles and operation of a system and a method for treating lice and nits on a head of a patient, according to the present invention may be better understood with reference to the drawings and the accompanying description.

Before explaining at least one embodiment of the invention in detail, it is to be understood that the invention is not limited in its application to the details of construction and the arrangement of the components set forth in the following description or illustrated in the drawings.

Unless otherwise defined, all technical and scientific terms used herein have the same meaning as commonly understood by one of ordinary skill in the art to which this invention belongs. The materials, dimensions, methods, and examples provided herein are illustrative only and are not intended to be limiting.

The following list is a legend of the numbering of the application illustrations:

11a rigid container

11b elastic container

12a inlet tube

12b gas release mechanism

12c connecting tube

12d connector 13 cap (airtight)

13a interior side of the cap

14 hair

15a mixture of gases (for treating lice)

15b gas

15c active material

16 space (airtight)

17 sealing means

18 unidirectional valve

20 gas distribution device

21 gas distribution device base

22 gas delivery pin

23 outlet hole

24 interior volume (of the gas distribution device base)

30 active material container

31 cork

32 coil

33 sponge

41 distributor

42a injection tube

42b injection tube stitch

42c injection tube edge

42d flow opening

43 vaporizing and circulating gas source

43g vaporizing and circulating gas

50 active agent container

51 container main wall

51a container main wall first end

51b container main wall second end

52 container end wall

53 cap

55 filter

56 filter compartment

56a filter compartment first disc

56b filter compartment second disc

56c filter compartment disc hole

57 seal

58 fluid passage

58a sealing disc

59 sponge

60 active agent

71-77 first method of treating lice and nits on a head of patient stages

81-86 second method of treating lice and nits on a head of patient stages

100a first embodiment of a system for treating lice and nits

100b second embodiment of a system for treating lice and nits

100c third embodiment of a system for treating lice and nits

100d fourth embodiment of a system for treating lice and nits

300 head

As used herein the specification and in the claims section that follows, the term “effective amount of an acidic active ingredient component” and the like refer to quantity and qualities of an active ingredient component sufficient to exterminate lice and nits on a patient's head. These can be determined according to experimental results.

The disclosed invention describes a system and method for treating lice on a patient's head. The invention utilizes a cap for defining the space around the treated hair, and an airtight sealing of the space around the treated hair, to apply a gas for treating lice. Reducing the volume that is treated allows using a small amount of gas, such that in case of releasing the gas outside the cap, the patient faces no danger due to the strong dilution of the gas. Keeping the gas compressed in a container enables a rapid delivery of the gas into the airtight space.

Additional options for delivery of the gas also include use of a flexible gas container which is pressed manually, a gas container built as a cylinder and piston which is pressed manually, or a container including a pump which can be activated manually or by means of a small engine, which could be electric for example.

This specification is not intended in any way to limit the present invention to any specific method of applying pressure to the material in order to deliver it in gas form.

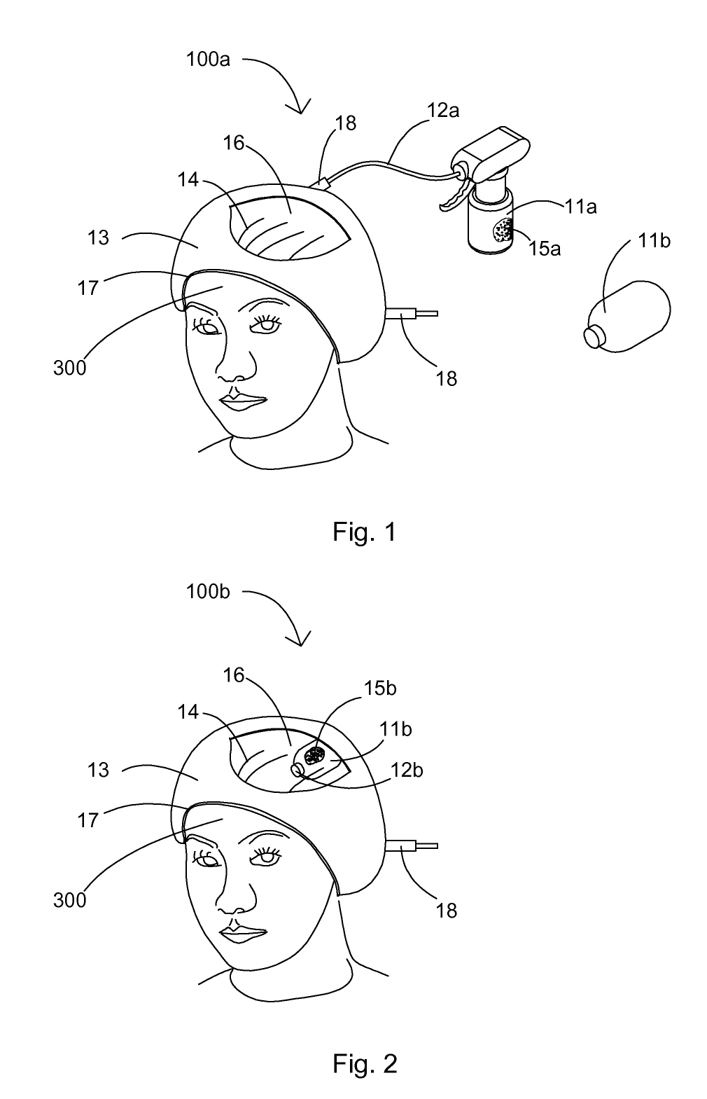

Referring now to the drawings, FIG. 1 illustrates a first embodiment of a system 100a, for treating lice and nits, on a head 300 of a patient, according to the present invention.

The first embodiment of a system 100a comprises an airtight cap 13 defining an airtight space 16 around head 300, a rigid container 11a for storing a mixture of gases 15a for treating lice and an inlet tube 12a for delivering mixture of gases 15a into airtight space 16 as an aerosol. Cap 13 is kept airtight by a sealing means 17 at the rim of cap 13. Mixture of gases 15a comprises at least one gas that causes irreversible damage to at least one essential mechanism of the lice. Rigid container 11a is located outside cap 13, and is coupled to cap 13 by inlet tube 12a, that comprises a tube.

If a segment of cap 13 is removed, and also if cap 13 is made of translucent material, the patient's hair 14 can be seen, as well as at least a part of airtight space 16, which is the volume between head 300 and hair 14, and the internal side of cap 13.

Airtight space 16 is not fixed, seeing as cap 13 can be made of material which is not rigid and therefore can change shape according to the regimen of pressures and forces applied to it.

Rigid container 11a contains a mixture of gases 15a which have a higher pressure than that of the environment, and thanks to the pressure difference, are delivered to the airtight space 16 in free flow, such as flow of fluid through an open tap. An additional option is storage of the mixture of gases 15a at a pressure of one atmosphere and delivering it when desired by means of activation of a pump or any other method, which causes it to flow.

In some embodiments, rather than a rigid container 11a, there is an elastic container 11b, which contains the mixture of gases 15a. In this case, the delivery of gas is done by pressure applied to compress the elastic container 11b.

The first embodiment of a system 100a can also include a unidirectional valve 18 which enables flow of air and mixture of gases 15a out of the airtight space 16 when the pressure rises above a predefined value, as well as a unidirectional valve 18 assembled to the inlet tube 12a, preventing the return of the mixture of gases 15a from the airtight space 16, to the rigid container 11b or to the elastic container 11b, according to the type of container used.

The mixture of gases 15a can include various concentrations of toxic gases, suffocating gases, or combinations of toxic and suffocating gases.

Examples of possible types of gasses are: vapors of acids such as acetic acid, hydrochloric acid and nitric acid, for attacking the respiratory system by attacking the soft tissues; carbon monoxide, for attacking the respiratory system by preventing oxygen from binding to the oxygen carrying protein of the lice; organophospates, for attacking the reproductive system; and insecticides belonging to organochlorines, carbamates, and organophosphates for attacking the nervous system.

FIG. 2 illustrates a second embodiment of a system 100b, for treating lice and nits, on a head 300 of a patient, according to the present invention.

The second embodiment of a system 100b comprises of an airtight cap 13 defining an airtight space 16 around head 300, a container which can be a rigid container 11a or an elastic container 11b, for storing a mixture of gases 15a for treating lice and a gas release mechanism 12b for delivering mixture of gases 15a into airtight space 16. Cap 13 is kept airtight by a sealing means 17 at the rim of cap 13. Mixture of gases 15a comprises at least one gas that causes irreversible damage to at least one essential mechanism of said lice. The gas release mechanism 12b is a release valve contained within the container. The container is attached to the inner side of cap 13.

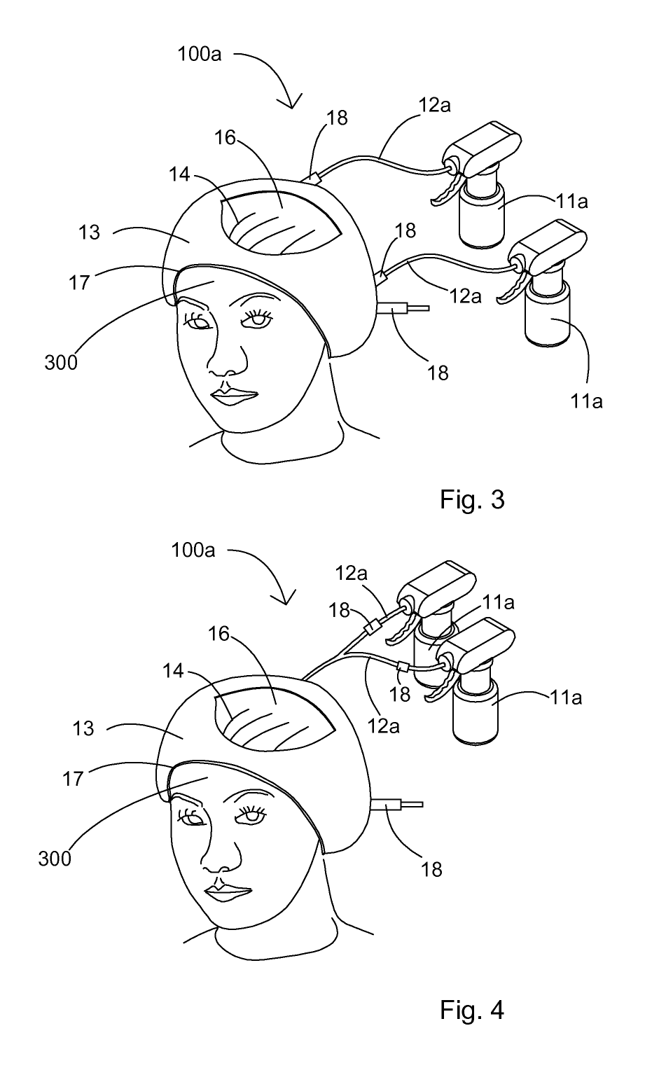

FIG. 3 illustrates the first embodiment of a system 100b, for treating lice and nits, on a head 300 of a patient, having two sets of rigid containers 11a, inlet tubes 12a, and unidirectional valves 18, according to the present invention. This configuration enables feeding two types of gasses or of mixtures of gasses into the airtight space 16 at the same time or at different times. When the feeding is done at different times, a suitable regimen of pressures can be used to press the first gas out of unidirectional valve 18 by means of the second gas. This valve is directed to enable only outward flow, while two additional unidirectional valves 18 are directed to prevent flow back into each one of both rigid containers 11a. Either of the rigid containers 11a can be replaced with an elastic container 11b.

FIG. 4 illustrates the first embodiment of a system 100b, for treating lice and nits, on a head 300 of a patient, having two sets of rigid containers 11a, unidirectional valves 18, and inlet tubes 12a, according to the present invention, while both of the inlet tubes 12a join into a single inlet tube, according to the present invention. This configuration also enables feeding two types of gasses or mixtures of gasses into the airtight space 16 at the same time or at different times. When the feeding is done at different times, a suitable regimen of pressures can be used to press the first gas out of unidirectional valve 18 by means of the second gas. This valve is directed to enable only outward flow, while two additional unidirectional valves 18 are directed to prevent flow back into each one of both rigid containers 11a. Either of the rigid containers 11a can be replaced with an elastic container 11b.

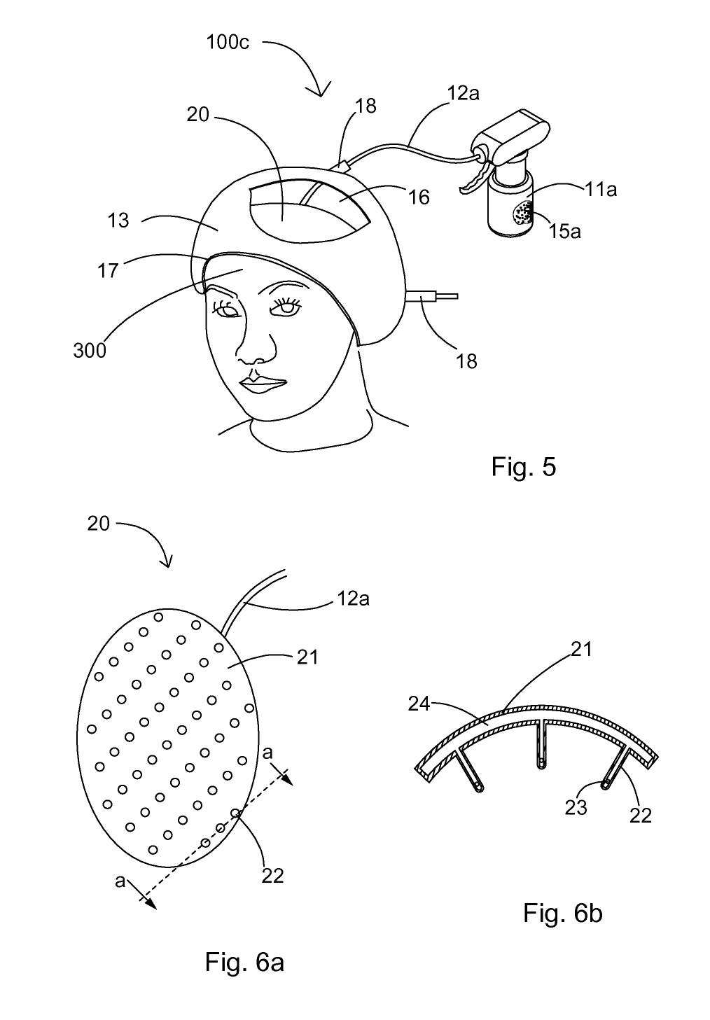

FIG. 5 illustrates the third embodiment of a system 100c, for treating lice and nits, on a head 300 of a patient, having a gas distribution device 20, according to the present invention.

The gas distribution device 20 serves for delivery and distribution under hair on a human scalp.

When gasses are intended to be used on human patient head as treatment of lice infestation, there are two functional problems:

(1) The gas needs to be prevented from escape away from around the head, and

(2) The gas needs to be preferentially applied in close proximity to the scalp where the lice mostly reside.

The solution to the first problem, as already outlined in the art, is to put a well-sealed cap over the head, and deliver the gas under the cap. Yet, as the gas is delivered from the top above the hair, only a minor fraction of it reaches under the hair in the proximity of the scalp skin (near the hair roots). Hence, such delivery of gas has a deficient effectiveness in treatment of lice infestation.

According to the present invention a gas distribution device 20 improve the effectiveness of gas treatments of lice infestations.

FIG. 6a illustrates a bottom view of a gas distribution device 20, according to the present invention, upon which the section plane a-a is marked. The present illustration shows a gas distribution device base 21, to the bottom of which are connected gas delivery pins 22.

FIG. 6b is a cross sectional view a-a schematic illustration of an exemplary, illustrative embodiment of a gas distribution device 20, according to the present invention.

The key element of the gas distribution device 20 is a series of gas delivery pins 22 with outlet holes 23 for gas or vapor delivery. Such gas delivery pins 22 may be short or elongated, stiff or flexible, with one or many outlet holes 23, etc.

Similar to the form of a brush, the gas delivery pins 22 are attached to and distributed over a common gas distribution device base 21. Here the gas distribution device base 21 has an interior volume 24. An inlet tube 12a is connected to the gas distribution device base 21. Hence, when vapor is pumped into the interior volume 24, through inlet tube 12a, it is then naturally fed into the connected gas delivery pins 22, and the gas is further on distributed out through the outlet holes 23. During use, this “brush” of pins is held in place pressed onto the scalp, such that the outlet holes 23 are under the hair in proximity to the scalp skin. Consequently, the gas is distributed as desired under the hair in proximity to the scalp skin.

In some embodiments, the end of said gas pins is rounded, and the outlet holes 23 are sideways from the shaft of the pins (as illustrated in the present figure).

The covering cap 13 is then put over the head and the gas distribution device 20, so as to prevent the delivered gas from escaping the proximity of the head. There are several options for engagement of a gas distribution device 20 in the third embodiment of a system 100c, for treating lice and nits. As was shown in FIG. 5, the inlet tube 12a, can go through the cap 13. In this case, the gas distribution device 20 and the cap 13 are a single unit and are placed together on the patient's head.

According to a second option, the gas distribution device 20 and the cap 13 are separated, the inlet tube 12a does not go through cap 13, and they are placed on the patient's head one after the other. In yet another option, the gas distribution device 20 comprises an integral part of the cap 13. Likewise, inlet tube 12a can be a continuous tube connecting the rigid container 11a, or the elastic container 11b, to the gas distribution device 20; or can be segmented, so that part of it is connected to the rigid container 11a, or the elastic container 11b, and its other part is connected to the gas distribution device 20, and only during application to the patient's head are they connected to each other to form a continuous inlet tube 12a.

According to some embodiments of the invention, the amount of mixture of gases 15a stored in the container, when released outside cap 13, is diluted by surrounding air such that the diluted mixture reaches non-toxic levels to the patient. The amount of mixture of gases 15a stored in the container is such that it would be highly toxic to the lice in a high concentration, and non-toxic to the patient if accidentally released into the surrounding air. The high concentration is achieved by defining and sealing the space 16 around the patient's head 300 and delivering mixture of gases 15a only into space 16.

According to some embodiments of the invention, the container holds mixture of gases 15a in a compressed state. Holding mixture of gases 15a in a compressed state allows a rapid release of mixture of gases 15a into airtight space 16.

According to some embodiments of the invention, cap 13 is a version of a swim cap, worn tightly on the patient's head. According to other embodiments of the invention, cap 15a is a version of a shower cap, worn loosely on the patient's head.

According to yet other embodiments of the invention, cap 13 is made in a version that is attractive in color and form to children. According to some embodiments of the invention, cap 13 is made of at least one of the following materials: silicone, rubber, polyethylene, latex, lycra, or cloth coated with a gas-tight layer.

According to some embodiments of the invention, exemplary composition of the gas mixture may include butane mixed with nitrogen, in a ratio ranging from 2:8 to 7:3 respectively. These gases are toxic.

According to some embodiments of the invention, the cap is configured to enable the mixture of gases to gradually flow outside of the cap to enhance safety.

According to some embodiments of the invention, exemplary duration of treatment ranges from 10 seconds to 30 minutes. According to some embodiments of the invention, the cap 13 is configured to comprise small holes enabling a slow escape of the gas mixture, or alternatively the cap is made of a fabric made to allow slow gas escape of gas to the outside and air to the inside.

According to some embodiments of the invention, cap 13 is attached to the patient's head and sealed by sealing means 17 for preventing mixture of gases 13 from escaping out of cap 13. According to some embodiments of the invention, sealing means 17 comprises of a rubber band on the rim of cap 13. Sealing cap 13 provides a higher effectiveness of applied mixture of gases 15a as well as enhanced user safety.

According to some embodiments of the invention, mixture of gases 15a comprises a gas with low molecular weight and high diffusivity. These properties are needed to allow a quick penetration of the gas to airtight space 16, below hair 14 and into the tracheal system of the lice. According to some embodiments of the invention, mixture of gases 15a comprises a gas having a molecular weight below 100 Dalton, for example gaseous hydrocarbons, NH₃, SO₂, CO₂, CO.

According to some embodiments of the invention, mixture of gases 15a comprises a gas having a molecular weight below 1,000 Dalton, for example amino acids and peptides.

According to some embodiments of the invention, mixture of gases 15a depletes oxygen from airtight space 16 by physical or chemical means. Such means may be inert gases like N₂ that displace oxygen with airtight space 16 and within the tracheae of the lice. Alternatively the oxygen depletion may be chemically achieved by a reaction between at least one of the gases in the mixture and the oxygen in the airtight space.

According to some embodiments of the invention, a unidirectional valve 18 is attached to cap 13. The unidirectional valve is configured to allowing air out of cap 13 but preventing air from entering cap 13. The unidirectional valve 18 further reduces the airtight space 16 around the head of the patient, particularly depleting oxygen out of airtight space. Using the unidirectional valve also enhances the affectivity of mixture of gases 15a by concentrating the fixed amount of mixture of gases 15a in container 11 to a smaller space 16.

According to some embodiments of the invention, mixture of gases 15a causes irreversible damage to at least one essential mechanism of the lice, such as the mitochondria, nervous system, reproductive system, feeding system, etc. According to some embodiments of the invention, mixture of gases 15a comprises of specific inhibitors or destroyers of enzymes or reactions related to the proper functioning of these systems. According to other embodiments of the invention, mixture of gases 15a comprises of compounds preventing copulation or compounds that render nits sterile.

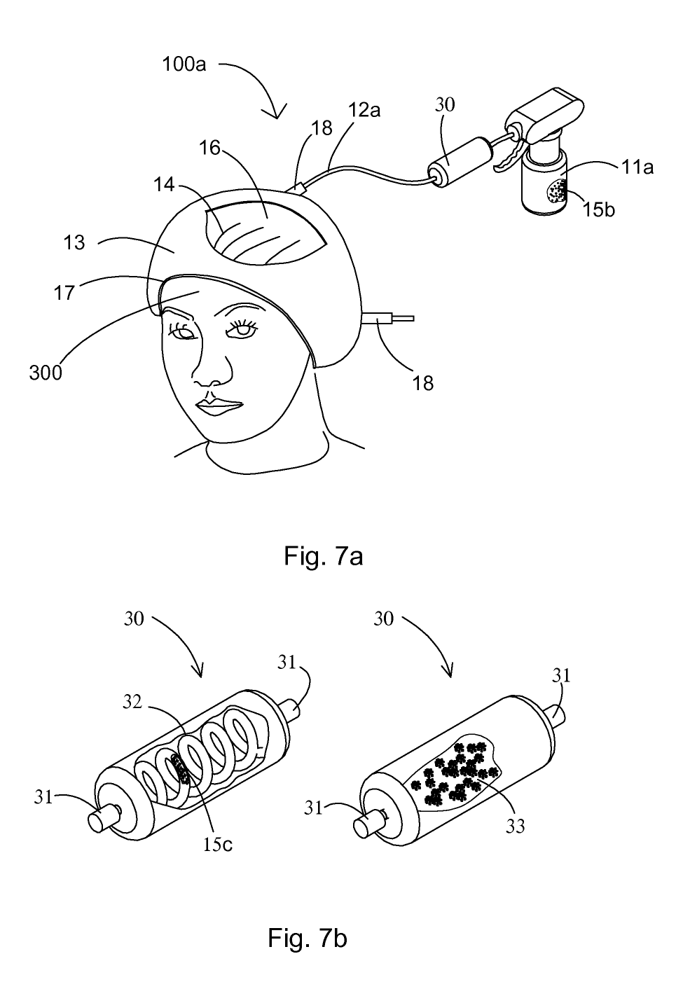

FIG. 7a illustrates the first embodiment of a system 100a, for treating lice and nits, on a head 300 of a patient, according to the present invention, in which the active material is stored separately within an active material container 30.

The active material container 30 is connected on both ends to inlet tube 12a. In this configuration, the rigid container 11a or elastic container 11b contains gas 15b at a sufficient pressure to maintain flow through the active material container 30 during use.

FIG. 7b shows two active material containers 30, according to the present invention. The right one contains a sponge 33 immersed with active material and the left one contains a coil 32 which contains an active material 15c. At both ends of the active material containers 30 shown here, there are two corks 31. This enables separate storage of the active material containers 30 containing active material 15c, and connection to inlet tube 12a prior to treatment of lice and nits.

The above description is not intended in any way to limit the present invention to these specific methods of storage and dispersion. This method of storing active material 15c separately from the carrying gas can be applied in any and all embodiments of the present invention.

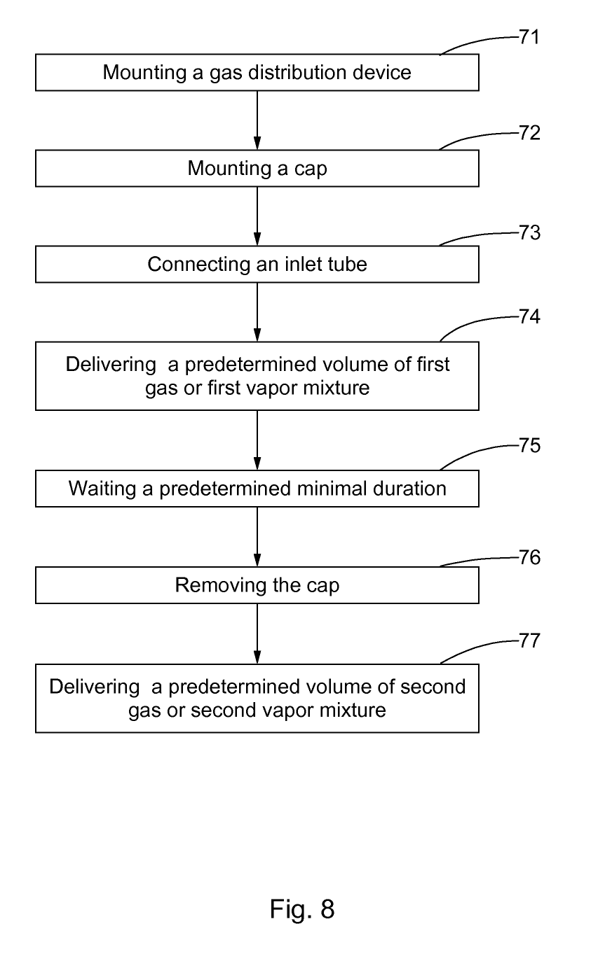

FIG. 8 is a block diagram schematically illustrating a first method of treating lice and nits on a head of a patient, according to some embodiments of the invention.

The method includes:
(a) mounting on the head of the patient a gas distribution device, the gas distribution device including a plurality of gas delivery pins, (stage 71);
(b) mounting a cap over the head of the patient, (stage 72);
(c) connecting an inlet tube part of the gas distribution device to an inlet part of the gas container, (stage 73);
(d) delivering through the gas distribution device a predetermined volume of a first gas or first vapor mixture containing effective amounts of gas or vapor for treatment of lice infestation, (stage 74);
(e) waiting at least a predetermined minimal duration, (stage 75); and
(f) removing the cap from the head after the predetermined minimal duration, (stage 76).

The method can further comprise:
(g) delivering, through the gas distribution device, a predetermined volume of second gas or second vapor mixture containing effective amounts of gas or vapor for treatment of lice infestation, (stage 77).

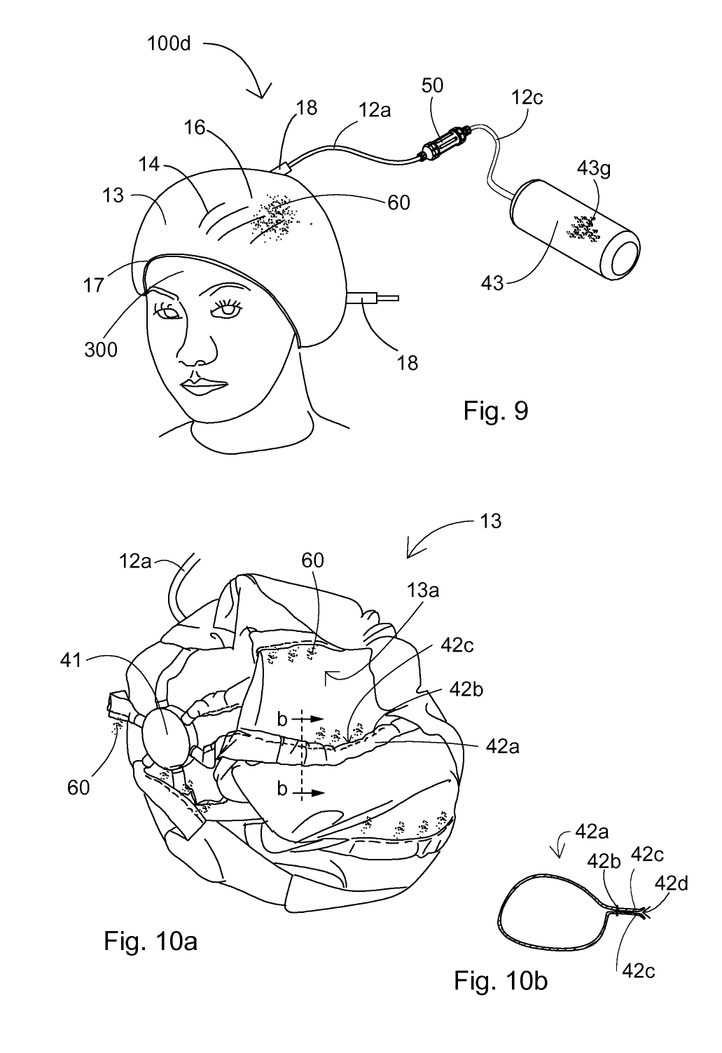

FIG. 9 illustrates a fourth embodiment of a system for treating lice and nits 100d, according to the present invention.

The fourth embodiment of a system for treating lice and nits 100d, according to the present invention includes cap 13, which is preferably made of a material impervious to active agent 60. Cap 13 can be equipped with a sealing means 17 to help prevent any leakage of active agent 60.

Cap 13 can be equipped with two unidirectional valves 18, one of which is connected to an inlet tube 12a. In the process of treatment, the inlet tube 12a is connected to an active agent container 50, which is connected to a vaporizing and circulating gas source 43. The present illustration shows a translucent area of cap 13, with the purpose of showing the space 16 between the patient's head 300 and hair 14 and the active agent 60 contained within the space 16.

The present illustration shows a translucent area of the vaporizing and circulating gas source 43 for the purpose of showing the contained vaporizing and circulating gas 43a. Prior to activation, the cap 13, the active agent container 50, and the vaporizing and vaporizing and circulating gas source 43 can be disconnected from each other. In preparation for treatment, they are connected as shown in the present illustration, and the vaporizing and circulating gas 43a causes evaporation of the active agent 60 which is contained within the active agent container 50, and circulates it through the cap 13.

The vaporizing and circulating gas source 43 can also be a pump, manual or electric, or a container containing vaporizing and circulating gas 43a at a pressure higher than one atmosphere.

The vaporizing and circulating gas 43a can also be air, or another gas such as N₂O or CO₂, or a mixture of them.

The active agent container 50 and the circulating gas source 43 can be connected to each other by means of connecting tube 12c, as shown in the present illustration, however they can be a unit, either composed as an integrated unit, or directly connected to each other. A typical example of the performance of the vaporizing and circulating gas source 43 is circulating one to five liters per minute of vaporizing and circulating gas 43a over three to ten minutes continuously or in intervals.

FIG. 10a illustrates a cap 13 of the fourth embodiment of the system for treating lice and nits 100d, according to the present invention, upon which a section plane b-b is marked.

Cap 13 is shown in the present illustration from its inside, with its central part pulled and diverted toward the left side of the illustration, and being equipped with a distributor 41. During treatment, the distributor 41 receives a supply of active agent 60 at a higher pressure than that of the environment.

The active agent 60 comes through the inlet tube 12a and the distributor 41 disperses its circulation to several injection tubes 42a. The injection tubes 42a are attached to the interior side of the cap 13a. An injection tube 42a includes openings through which the active agent 60 flows.

FIG. 10b is a cross sectional view b-b illustration of an injection tube 42a, of the cap 13 of the fourth embodiment of the system for treating lice and nits 100d, according to the present invention.

The injection tube 42a can be composed of a thin material, relative to its lateral section, for example fabric, folded double lengthwise. Along the injection tube edge 42c are injection tube stitches 42b, which connect these edges however do not seal them, and comprise a flow opening 42d.

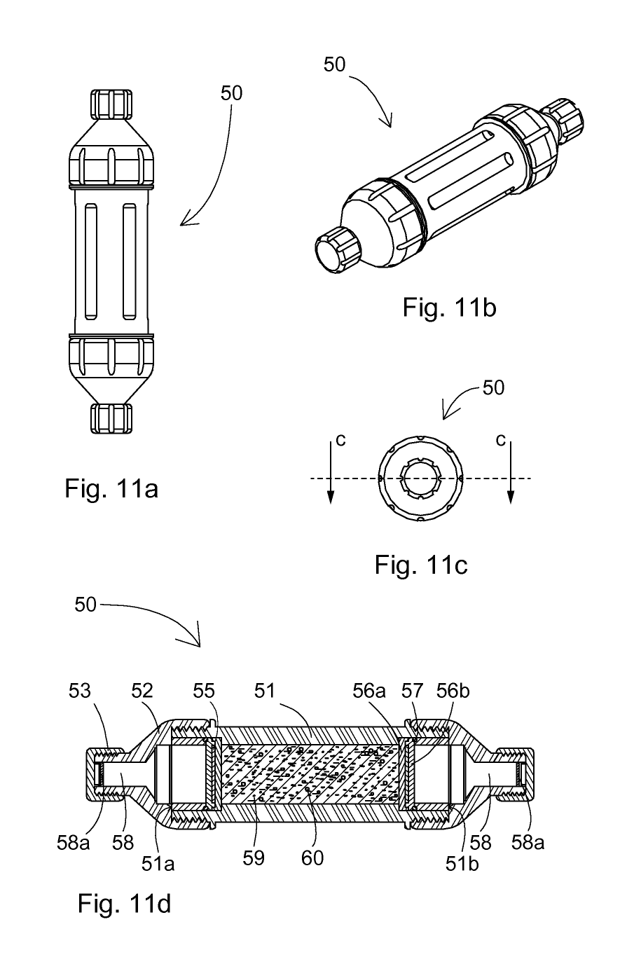

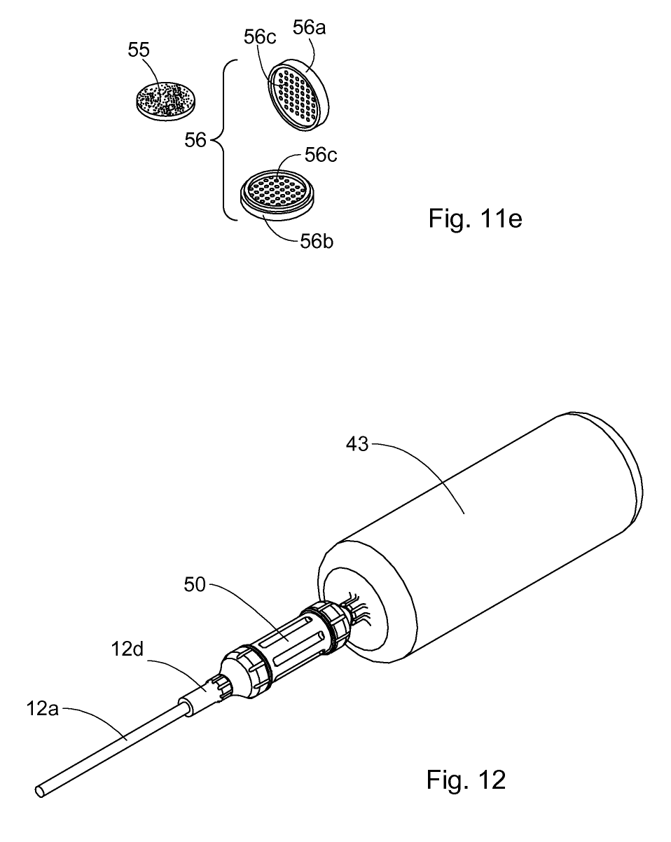

FIG. 11a is a side view schematic illustrations of an active agent container 50 of the fourth embodiment of the system for treating lice and nits 100d, according to the present invention.

FIG. 11b is an isometric view schematic illustration of an active agent container 50 of the fourth embodiment of the system for treating lice and nits 100d, according to the present invention.

FIG. 11c is a top view schematic illustration of an active agent container 50 of the fourth embodiment of the system for treating lice and nits 100d, according to the present invention, upon which a section plane c-c is marked.

FIG. 11d is a cross sectional view c-c illustration of an active agent container 50 of the fourth embodiment of the system for treating lice and nits 100d, according to the present invention.

The present illustration shows the active agent container 50, in storage state, when it is inactive, and contains active agent 60 in a liquid state, absorbed within a sponge 59. The sponge 59 can be a single lump, several smaller lumps, or a large number of small crumb-size pieces, spherical or of another shape, including amorphous pieces. The pieces of the sponge 59 improve the evaporation, and prevent flow of the active agent 60, when it is in liquid state.

The active agent container 50 can be designated for single-time use.

A particularly effective form of the active agent 60 for this specific purpose is acetic acid, (CH₃COOH).

A sufficient quantity of acetic acid for one treatment, stored in active agent container 50, can be in the range of 2 milliliters to 5 milliliters.

During circulation of the vaporizing and circulating gas source 43, (not shown in the present illustration, shown in FIG. 9), the active agent 60 evaporates, and only its vapors emerge and flow from the active agent container 50.

The active agent container 50 includes a container main wall 51, whose shape can be as of a cylindrical tube wall, with a circular section, having at each of its ends a container end wall 52, the shape of each can include a conical segment, which tapers and becomes narrower in the outward direction, having a circular section, which extends into a segment resembling a cylindrical tube wall, with a circular section, creating at it center a fluid passage 58. One fluid passage 58 serves as an inlet, while the other serves as an outlet.

In storage state, the active agent container 50 is closed on both ends by caps 53 which are removed from their places prior to use. Near the end of each fluid passage 58, a sealing disc 58a can be placed to prevent any leakage of active agent 60 during storage.

The sealing discs 58a are broken during preparation for use, for example during assembly of a connector 12d, (not shown in the present illustration, shown in FIG. 12).

Furthermore, the active agent container 50 can include two seals 57 and two filters 55.

FIG. 11e is an isometric view schematic illustration of filter compartment 56 and a filter 55, of the active agent container 50, of the fourth embodiment of the system for treating lice and nits 100d, according to the present invention.

The present illustration shows one possible configuration for installing the filter 55 in its place, including a filter compartment 56 composed of two parts, a filter compartment first disc 56a, and a filter compartment second disc 56b each of which has filter compartment disc holes 56c, with the filter 55 disposed between them.

FIG. 12 is an isometric view schematic illustration of a vaporizing and circulating gas source 43, an active agent container 50, and an inlet tube 12a, of the fourth embodiment of the system for treating lice and nits 100d, according the present invention.

The present illustration presents a vaporizing and circulating gas source 43 which is connected directly to the active agent container 50. Likewise, the inlet tube 12a is also connected to the active agent container 50, a connection made, in the case of the present illustration, by means of connector 12d.

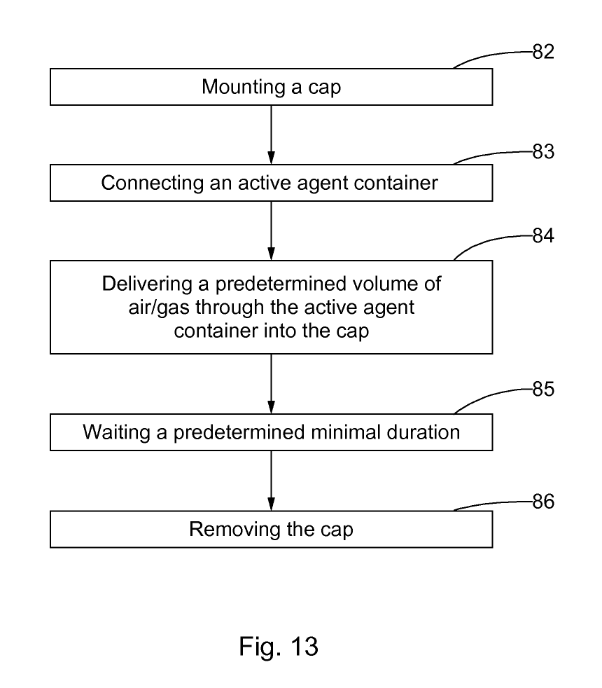

FIG. 13 is a block diagram schematically illustrating of a second method of treating lice and nits on a head of a patient, according to some embodiments of the present invention.
(a) mounting a cap over the head of the patient, the cap includes an inlet tube, a distributor and injection tubes, (stage 82);
(b) connecting an active agent container to the inlet tube, the active agent container includes an active agent in liquid state absorbed in a in sponge, (stage 83);
(c) delivering vaporizing and circulating gas through the active agent container, and evaporating the active agent, the gas may be air or another suitable gas, or a mixture of suitable gases, (stage 84);
(d) waiting at least a predetermined minimal duration, (stage 85); and
(e) removing the cap from the head after the predetermined minimal duration, (stage 86).

In the above description, an embodiment is an example or implementation of the inventions. The various appearances of “one embodiment,” “an embodiment” or “some embodiments” do not necessarily all refer to the same embodiments.

Although various features of the invention may be described in the context of a single embodiment, the features may also be provided separately or in any suitable combination. Conversely, although the invention may be described herein in the context of separate embodiments for clarity, the invention may also be implemented in a single embodiment.

It is understood that the phraseology and terminology employed herein is not to be construed as limiting and are for descriptive purpose only.

It is to be understood that the details set forth herein do not construe a limitation to an application of the invention.

Furthermore, it is to be understood that the invention can be carried out or practiced in various ways and that the invention can be implemented in embodiments other than the ones outlined in the description above.

It is to be understood that where the claims or specification refer to “a” or “an” element, such reference is not be construed that there is only one of that element.

It is to be understood that where the specification states that a component, feature, structure; or characteristic “may”, “might”, “can” or “could” be included, that particular component, feature, structure, or characteristic is not required to be included.

Where applicable, although state diagrams, flow diagrams or both may be used to describe embodiments, the invention is not limited to those diagrams or to the corresponding descriptions. For example, flow need not move through each illustrated box or state, or in exactly the same order as illustrated and described. Methods of the present invention may be implemented by performing or completing manually, automatically, or a combination thereof, selected steps or tasks. The term “method” may refer to manners, means, techniques and procedures for accomplishing a given task including, but not limited to, those manners, means, techniques and procedures either known to, or readily developed from known manners, means, techniques and procedures by practitioners of the art to which the invention belongs.

The descriptions, examples, methods and materials presented in the claims and the specification are not to be construed as limiting but rather as illustrative only.

Meanings of technical and scientific terms used herein are to be commonly understood as by one of ordinary skill in the art to which the invention belongs, unless otherwise defined.

The present invention can be implemented in the testing or practice with methods and materials equivalent or similar to those described herein.

Any publications, including patents, patent applications and articles, referenced or mentioned in this specification are herein incorporated in their entirety into the specification, to the same extent as if each individual publication was specifically and individually indicated to be incorporated herein. In addition, citation or identification of any reference in the description of some embodiments of the invention shall not be construed as an admission that such reference is available as prior art to the present invention.

While the invention has been described with respect to a limited number of embodiments, it will be appreciated that many variations, modifications and other applications of the invention may be made.

1. A system for treating lice and nits on a head of a patient, said system comprising:
(a) an active agent container for storing a predetermined volume of active agent, wherein said active agent container includes:
(i) a container main wall having a container main wall first end and a container main wall second end; and
(ii) two container end walls, disposed on said container main wall first end and said container main wall second end,
wherein said system for treating lice and nits has a storage state and active state.

2. The system for treating lice and nits of claim 1, wherein said system for treating lice and nits is in said storage state, wherein said active agent container store an active agent, wherein said active agent is in a liquid state.

3. The system for treating lice and nits of claim 2 wherein said active agent container store a sponge, wherein said active agent is absorbed by said sponge.

4. The system for treating lice and nits of claim 2 wherein said active agent container store a plurality of sponge pieces, wherein said active agent is absorbed by said sponge pieces.

5. The system for treating lice and nits of claim 4, wherein said active agent is an acetic acid.

6. The system for treating lice and nits of claim 4, wherein said active agent container further includes:
(iii) at least one filter disposed inside of said active agent container;
(iv) two lids mounted on said two container end walls.

7. The system for treating lice and nits of claim 6, wherein said active agent is an acetic acid, and wherein said active agent has a volume of at least two milliliters.

8. The system for treating lice and nits of claim 7, wherein said active agent container further includes:
(v) at least one sealing disc disposed inside said active agent container.

9. The system for treating lice and nits of claim 1, wherein said system for treating lice and nits is in said active state, wherein said active agent container store an active agent, wherein said active agent is partially in a liquid state and partially in a gas state.

10. The system for treating lice and nits of claim 9 further comprising:
(b) a cap, wherein said cap is operatively connected to said active agent container.

11. The system for treating lice and nits of claim 9 further comprising:
(b) an inlet tube wherein said inlet tube is connected to said active agent container, and
(c) a cap, wherein said cap is operatively connected to said inlet tube.

12. The system for treating lice and nits of claim 10 further comprising:
(d) a vaporizing and circulating gas source, wherein said a vaporizing and circulating gas source is operatively connected to said active agent container.

13. The system for treating lice and nits of claim 12, wherein said active agent is an acetic acid.

14. The system for treating lice and nits of claim, wherein said cap is made from a material that is practically impenetrable to said active agent, wherein said cap has an interior side.

15. The system for treating lice and nits of claim, wherein said cap includes:
(i) a distributor;
(ii) at least two injection tubes, wherein each one of said injection tubes is operatively connected to said injection tubes, wherein each one of said injection tubes is disposed on said interior side of said cap, and wherein each one of said injection tubes has plurality of flow openings.

16. The system for treating lice and nits of claim 15 wherein said active agent is an acetic acid.

17. The system for treating lice and nits of claim 15 wherein there is a flow of active agent from said active agent container through said distributor, through said injection tubes and through said flow opening.

18. The system for treating lice and nits of claim 16 wherein there is a flow of active agent from said active agent container, through said distributor, trough said injection tubes and through said flow opening.

19. The system for treating lice and nits of claim 18 wherein there is a continues flow of a vaporizing and circulating gas from said a vaporizing and circulating gas source through said active agent container, at a rate of at least one liter per minute for at least three minutes.

20. A method for treating lice and nits on a head of a patient the method comprising the stages of:
(a) mounting a cap over the head of said patient, said cap 1 includes an inlet tube, a distributor and injection tubes;
(b) connecting an active agent container to said inlet tube, wherein said active agent container includes an active agent in liquid state absorbed in a in sponge;
(c) delivering a vaporizing and circulating gas through the active agent container, aid evaporating the active agent;
(d) waiting at least a predetermined minimal duration; and
(e) removing said cap from said head after the predetermined minimal duration.

21. The method for treating lice and nits on a head of a patient of claim 20, wherein said active agent is an acetic acid.

22. The method for treating lice and nits on a head of a patient of claim 20, wherein there is a flow of said active agent from said active agent container, through said distributor, and through said injection tubes.
